(19) United States
(12) Patent Application Publication
(10) Pub. No.: US 2005/0154307 A1
(43) Pub. Date: Jul. 14, 2005

(54) Apparatus and method for measuring a fluid velocity profile using acoustic doppler effect

(76) Inventors: Noritomo Hirayama, Tokyo (JP); Toshihiro Yamamoto, Tokyo (JP); Hironobu Yao, Tokyo (JP)

Correspondence Address: Marc A. Rossi;ROSSI & ASSOCIATES, P.O. Box 826, Ashburn, VA, 20146-0826, US

(21) Appl. No.: 10/999,224

(22) Filed: Nov. 29, 2004

(30) Foreign Application Priority Data
Nov. 27, 2003 (JP) 2003-396755

(51) Int. Cl. A61B 8/06

(52) U.S. Cl. 600/453

(57) ABSTRACT

A clamp-on type acoustic Doppler current profiler eliminates, among ultrasound echoes caused by two measurement lines of a longitudinal wave and a shear wave propagating in a piping, the ultrasound echo based on the longitudinal wave, thereby providing the measurement of a flow rate profile and/or a flow rate with a higher accuracy. The profiler includes a wedge mounted to the piping. The wedge includes an inclined surface at which an ultrasonic transducer can be mounted. The inclination is such that the ultrasound transducer receives only the ultrasound echo from the reflection of a shear wave component off the reflectors in the fluid.

### BACKGROUND

A clamp-on type ultrasound flow meter typically uses an ultrasound transducer attached to a part of an outer periphery of a tubular body, such as a piping, to measure, from the exterior of the tubular body, the flow rate of fluid moving in the tubular body. Clamp-on type ultrasound flow meters are mainly classified into ones that utilize the difference in propagation time and ones that utilize the Doppler effect.

The former ones based on the difference in propagation time reciprocate ultrasound, applied slantedly across the tubular body to the fluid moving in the tubular body. The difference between the time in which the ultrasound propagates along the outward route and the time in which the ultrasound propagates along the return route is used to measure the fluid flow rate.

On the other hand, the latter ones, based on the Doppler effect, relies on reflectors, namely suspended particles and/or air bubbles included in the fluid, which are assumed to move at the same speed as the fluid. The movement speed of the reflectors is used to measure the flow rate of the fluid. Specifically, this technique transmits ultrasound into the fluid being measured, and the frequency of the ultrasound is changed by the Doppler effect in accordance with the speed of the reflectors when the ultrasound is reflected off the same. The frequency of the reflected ultrasound is detected to measure the speed of the reflectors, thereby measuring the fluid flow rate profile and/or the fluid flow rate.

A conventional Doppler ultrasound flow meter is disclosed, for example, in Japanese Laid-Open patent Publication No. 2000-97742. FIG. 6 schematically illustrates a structure of such a flow meter. The Doppler ultrasound flow meter shown in FIG. 6 includes an ultrasound velocity profile measurement unit (hereinafter referred to as a UVP unit) 10 that measures the flow rate of fluid 22 in a piping 21 in a non-contact or non-invasive manner. This UVP unit 10 includes an ultrasound transmission means 11 for transmitting to the fluid 22 an ultrasound pulse having a required frequency (basic frequency fₒ) along a measurement line ML, a flow rate profile measurement circuit 12 for receiving the ultrasound echo reflected from the measurement region of the ultrasound pulse transmitted into the fluid 22 to measure the flow rate profile thereof in the measurement region, a computer 31 (e.g., microcomputer, CPU, MPU) for calculating the flow rate profile of the fluid 22 to integrate it in the radius direction of the piping 21, thereby measuring the flow rate of the fluid 22 depending on time, and a display apparatus 32 for displaying the output from this computer 31 in chronological order.

The ultrasound transmission means 11 includes, for example, a signal generator 15, namely consisting of an transducer 13 for generating an electric signal having a basic frequency (e.g., 1 MHz, 2 MHz, 4 MHz) and an emitter 14 for outputting an electric signal from the transducer 13 as a pulse having a frequency F_{rpf} for each predetermined cycle (1/F_{rpf}). The signal generator 15 inputs a pulsed electric signal having the basic frequency F_{rpf} to the ultrasound transducer 16. The ultrasound transducer 16 transmits an ultrasound pulse having the basic frequency fₒ to the fluid 22 in the piping 21 along the measurement line ML. This ultrasound pulse is a straight beam having a beam width, for example, of 5 mm having very little dispersion.

The ultrasound transducer 16 also works as a transmitter/receiver that is designed to receive an ultrasound echo generated when a transmitted ultrasound pulse is reflected off the reflectors in the fluid 22. The reflectors can be air bubbles or suspended particles uniformly distributed in the fluid 22, i.e., foreign matter having different acoustic impedance from that of the fluid 22.

The transducer 16 converts ultrasound echo received thereby into an electric echo signal. This electric echo signal is amplified by an amplifier 17 in the UVP unit 10 and digitized by an AD converter 18. The digital echo signal is input into the flow rate profile measurement circuit 12. The electric signal having the basic frequency fₒ from the transducer 13 and digitized by the AD converter 18 is input to the flow rate profile measurement circuit 12. Based on the difference of frequency between these signals, the flow rate based on Doppler shift is measured to calculate the flow rate profile of the fluid 22 in the measurement region, along the measurement line ML. The flow rate profile of this measurement region can be corrected by the oblique angle α of the ultrasound transducer 16 (oblique angle to the direction perpendicular to the longitudinal or axial direction of the piping 21), thereby measuring the flow rate profile of the fluid 22 in the cross section of the piping 21.

Next, how the Doppler ultrasound flow meter operates will be further described in detail with reference to FIG. 7. In section (A) of FIG. 7, the ultrasound transducer 16 is inclined by the oblique angle α to the direction along which the fluid 22 flows. The ultrasound transducer 16 transmits the ultrasound pulse having the basic frequency fₒ into the piping. The ultrasound pulse collides with and is reflected off the reflectors (e.g., suspended particles uniformly dispersed in the fluid 22 on the measurement line ML), namely turning into an ultrasound echo “a,” which is received by the ultrasound transducer 16, as shown in section (B) of FIG. 7.

In section (B) of FIG. 7, reference numeral “b” denotes a multiple reflection echo reflected off the tubular wall of the piping 21 into which an ultrasound pulse is transmitted, and reference numeral “c” denotes a multiple reflection echo created at the tubular wall of the opposing section of the piping 21. The ultrasound transducer 16 transmits an ultrasound pulse having a cycle of (1/F_{rpf}) as shown. The echo signal “a” received by the ultrasound transducer 16 is filtered and the Doppler shift method is used to measure the flow rate profile along the measurement line ML, thereby providing the display as shown in section (C) of FIG. 7. This flow rate profile is measured by the flow rate profile measurement circuit 12 of the UVP unit 10 and is displayed by a display apparatus 32 via the computer 31.

As described above, the Doppler shift method uses a mechanism in which, when an ultrasound pulse is transmitted to the fluid 22 flowing in the piping 21, it is reflected off the reflectors mixed in or uniformly dispersed in the fluid 22, which turns into an ultrasound echo. The frequency of the ultrasound echo is shifted in a magnitude proportional to the flow rate. The flow rate profile signal of the fluid 22 measured by the flow rate profile measurement circuit 12 is transmitted to the computer 31 and the flow rate profile signal can be integrated in the radius direction of the piping 21, thereby calculating the flow rate of the fluid 22. The flow rate “m(t)” of the fluid 22 at time “t” can be represented by the following mathematical expression (1):
m(t)=ρ∫v(x·t)·dA (1),
where “ρ” represents the density of the fluid, “v(x·t)” represents the velocity component (in direction “x”) at time “t” and “A” represents the sectional area of the piping.

The above flow rate m(t) can also be calculated by the following mathematical expression (2):
m(t)ρ∫∫vx(r·θ·t)·r·dr·dθ (2),
where “vx(r·θ·t)” represents the velocity component at time “t” from the center on the cross section of the piping axis direction for distance “r” and angle “θ.”

To accurately determine the flow rate of the fluid 22 in both the steady state and the non-steady state by the above-described conventional Doppler ultrasound flow meter, the flow rate profile of the fluid 22 in the piping 21 must be detected accurately. As can be seen from the above-described measurement mechanism, the flow rate profile of the fluid 22 is obtained by subjecting the ultrasound echo off the reflectors in the fluid 22 to signal processing for calculation. For this reason, this ultrasound echo must contain only an acoustic signal. The acoustic and electric noise components must be eliminated.

Acoustic noise having an influence on this ultrasound echo includes for example that caused by the reflection or scattering between the mediums having different acoustic impedances and that caused by longitudinal and shear waves generated in solid matter (e.g., piping material). Solid matter (e.g., metal) generally includes therein two types of acoustic waves. One is called a compressional wave, a longitudinal wave having a displacement in the same direction as the direction along which a wave propagates, and the other is called a shear wave, a shear wave having a displacement in the direction perpendicular to the direction along which the wave propagates.

According to a publication entitled INTRODUCTION TO ELECTRIC ACOUSTIC ENGINEERING by SHOKODO Co., Ltd., pp. 247-251, when an acoustic wave is transmitted from a fluid into a solid matter in an oblique direction, the solid matter includes therein not only a longitudinal wave but also a shear wave. It is generally known that, when an acoustic wave propagates from one type of solid to another type of solid, then both the longitudinal and shear waves are caused along both the direction along which the acoustic wave is transmitted and the direction along which the acoustic wave is reflected.

How an ultrasound echo is influenced by a longitudinal wave and a shear wave in solid matter will be described with respect to FIG. 8. As shown in FIG. 8, an acoustic wave propagates from medium 1 to medium 2. In this case, the relation between a propagation angle θᵢₙ (incidence angle at the interface between both mediums) and an angle θₒᵤₜ (refraction angle or output angle at the boundary between both mediums) of the acoustic wave in mediums 1 and 2 can be expressed by the following mathematical expression (3):
sin θᵢₙ/c₁=sin θₒᵤₜ/c₂ (3),
where “c₁” represents the acoustic velocity in medium 1, “c₂” represents the acoustic velocity in medium 2, “θᵢₙ” represents an angle at medium 1(incidence angle), and “θₒᵤₜ” represents an angle at medium 2 (refraction angle).

When the acoustic wave from medium 1 is incident on medium 2 and the acoustic velocity c₂ in medium 2 is higher than acoustic velocity c₁ in medium 1 (c₁<c₂), there is a critical angle at which the acoustic wave is totally reflected at the interface between these mediums. This critical angle θ_{c} is represented by the following mathematical expression (4):
θ_{c}=sin⁻²(c₁/c₂) (4),
where “c₁” represents the acoustic velocity in medium 1, and “c₂” represents the acoustic velocity in medium 2, and c₁<c₂.

The following section will describe the oblique angle of the ultrasound transducer 16 of the conventional Doppler ultrasound current profiler shown in FIG. 6 (incidence angle of ultrasound to piping 21) in accordance with a publication (hereafter Publication 2) entitled DEVELOPMENT OF FLOW MEASUREMENT METHOD USING ULTRASONIC VELOCITY PROFILER (UVP) (6) NIST (US.), CALIBRATION: FLOW MEASUREMENT USING LOOPS—TEST RESULTS AND PRECISION VERIFICATION,” by Atomic Energy Society of Japan, 1999 autumn convention, 2001, and a publication (hereafter Publication 3) entitled DEVELOPMENT OF A NOVEL FLOW METERING SYSTEM USING ULTRASONIC VELOCITY PROFILE MEASUREMENT, Experiments in Fluids, vol. 32, 2003, pp. 153-160.

Publication 2 describes an example in which a so-called clamp-on type Doppler ultrasound current profiler is provided at an outer wall of a stainless piping for measuring the fluid flow rate. In this example, the ultrasound transducer has an oblique angle of 5 or 10 degrees. Publication 3 describes that an ultrasound transducer driven with a frequency of 1 MHz to the piping at an oblique angle of 5 degrees while an ultrasound transducer driven with a frequency of 4 MHz to the piping at an oblique angle of 0 to 20 degrees, and also describes that the ultrasound transducer and the piping have therebetween an acrylic member having a thickness of 2 mm to be used as a wedge.

FIG. 9 shows the structure in accordance with the measurement conditions described in Publication 3. Here, an acrylic wedge 42 is fixed with an ultrasound transducer 41 such that this ultrasound transducer 41 is inclined, at an angle θᵢₙ, relative to a direction perpendicular to the longitudinal direction of a piping 43. Specifically, ultrasound from the wedge 42 to the piping 43 has an incidence angle of θᵢₙ. According to Publications 2 and 3, the fluid 44 (as shown in FIG. 9) is water, while the piping 43 is made of stainless steel. The velocity of sound in water is about 1,500 m/s, while the velocity of the longitudinal wave in stainless steel is about 5,750 m/s and the velocity of the shear wave in stainless steel is about 3,206 m/s. The velocity of the longitudinal wave in acrylic is 2,730 m/s.

The critical angles θ_{c} of the longitudinal wave and the shear wave are calculated based on the above-described mathematical expression 4. The critical angle of the longitudinal wave at the interface between the wedge 42 and the piping 43 is 28.3 degrees, and the critical angle of the shear wave at the interface between the wedge 42 and the piping 43 is 58.4 degrees. When the ultrasound transducer 41 transmits an acoustic wave having an oblique angle (incidence angle) θᵢₙ of 20 degrees for example, the wedge 42 and the piping 43, both of which are solids, have a longitudinal wave and a shear wave at the interface therebetween. The incidence angle θᵢₙ at the above interface is equal to or lower than the critical angles of both of the longitudinal wave and the shear wave. Thus, the piping 43 has therein the propagation of both of the longitudinal wave and the shear wave.

Furthermore, the longitudinal wave and the shear wave propagating in the piping 43 are transmitted into water while being refracted. This causes two measurement lines ML. In the piping 43 shown in FIG. 9, the longitudinal wave has a refraction angle (output angle) θₚₗ of 46.1 degrees while the shear wave has a refraction angle θₚₛ of 23.7 degrees. When the acoustic wave is transmitted from the piping 43 into water, the acoustic wave is converted to a longitudinal wave and the refraction angle θ_{fl} in water is 10.84 degrees. A publication (hereafter Publication 4) entitled ULTRASONICS MANUAL, Editorial Committee of the Ultrasonics Manual, Maruzen Co., Ltd., discloses the transmission rate of the acoustic wave when, as in the above case, the acoustic wave is transmitted from metal into water. Here, medium 1 is made of aluminum while medium 2 is water (referring to FIG. 8).

FIG. 10 illustrates the relation shown in Publication 4 when an aluminum plate corresponding to the medium 1 and water corresponding to the medium 2 have a shear wave at the interface therebetween, between the incidence angle and the energy reflection coefficient (reflectivity) and the energy transmission coefficient (transmission rate). In FIG. 10, the wave “SV” represents a shear wave and the wave “L” represents a longitudinal wave. As can be seen from FIG. 10, total reflection does not occur and the longitudinal wave is transmitted even when the incidence angle of the shear wave exceeds 28 degrees. FIG. 11 shows the relation, when the aluminum plate and water have a longitudinal wave at the interface therebetween, between the incidence angle and the reflectivity and the transmission rate. As can be seen from FIG. 11, only the longitudinal wave is transmitted.

Next, FIG. 12 shows the behavior of the ultrasound echo in the structure of FIG. 9. The ultrasound echo from the reflector in water returns from water to the ultrasound transducer 41 via the same route as that through which ultrasound is transmitted from the piping 43 into water. The ultrasound echo has an incidence angle θ_{f} of 10.84 degrees when the ultrasound is transmitted from water into the aluminum piping 43. Thus, both the longitudinal wave and the shear wave are generated, as can be seen from FIG. 12.

As shown in FIG. 12, when the ultrasound echo is transmitted from water into the piping 43, two measurement lines of the longitudinal wave and the shear wave are generated, and thus, the piping 43 has therein four ultrasound echoes. When the ultrasound echo is transmitted from the piping 43 into the wedge 42, the acoustic wave has refraction in accordance with mathematical expression 3 but there is no critical angle because the wedge 42 is made of a material having a lower acoustic velocity than that of the piping 43. As a result, no total reflection occurs and four ultrasound echoes progress in the wedge 42 in the direction of the ultrasound transducer 41. Thus, the four ultrasound echoes propagating in the wedge 42 are transmitted into the ultrasound transducer 41 with a time difference in accordance with the acoustic velocity of the ultrasound transducer 41 in the propagation route.

In FIG. 12, “θₚₗ” represents the refraction angle of the longitudinal wave at the interface between the fluid (water) 44 and the piping 43, “θₚₛ” represents the refraction angle of the shear wave, “θ_{wl}” represents the refraction angle of the longitudinal wave at the interface between the piping 43 and the wedge 42, and “θ_{ws}” represents the refraction angle of the shear wave. The ultrasound echo received by the ultrasound transducer 41 has a time axis corresponding to the position along the direction of the diameter of the piping 43. The longitudinal wave and the shear wave in the piping 43 have different acoustic velocities. Thus, the ultrasound echo received by the ultrasound transducer 41 at a specific time is obtained by synthesizing the flow rate at point “A′” of the fluid 44 in the piping 43 measured by the shear wave in FIG. 13 with the flow rate at point “A′” (which is at a different position from that of point “A” along the direction of the diameter of the piping 43) of the fluid 44 in the piping 43 measured by the longitudinal wave.

Specifically, as schematically shown in FIG. 14, the flow rate calculated based on the ultrasound echo received by the ultrasound transducer 41 at a specific time is actually obtained by synthesizing the flow rates at point “A” and point “A′” (which are at different positions), and thus, the flow rate profile and consequently the flow rate of the fluid 44 in the piping 43 cannot be measured accurately.

As described above, the Doppler ultrasound flow meter for calculating the flow rate by measuring the flow rate profile in the piping has a problem in that an acoustic wave transmitted from the ultrasound transducer generates a longitudinal wave and a shear wave in a piping and the two measurement lines are transmitted into the fluid, which causes the ultrasound echoes from the respective reflectors to be received by the Doppler ultrasound flow meter, thus causing the flow rate profile to be measured inaccurately.

In view of the above problem discovered by the present inventors, there remains a need to provide an apparatus and a method for more accurately measuring the fluid flow rate profile and the fluid flow rate. The present invention addresses this need. Specifically, the above noted problems can be solved by eliminating or isolating, from the ultrasound echoes caused by two measurement lines of a longitudinal wave and a shear wave propagating in the tubular body (e.g., piping), the ultrasound echo caused by the longitudinal wave.

### SUMMARY OF THE INVENTION

The present invention relates to an apparatus and a method for measuring a fluid flow rate profile using a Doppler effect.

One aspect of the present invention is the apparatus, such as a clamp-on type acoustic Doppler current profiler, for measuring the flow rate profile of fluid traveling through a tubular body, based on the frequency of ultrasound changed by the Doppler effect when ultrasound is reflected off reflectors existing in the fluid. The tubular body is made of material that allows an acoustic wave to propagate therethrough. The profiler includes a wedge that can be externally mounted to the tubular body and an ultrasound transducer mounted to the wedge. The wedge also is made of material that allows an acoustic wave to propagate therethrough. The ultrasonic transducer is fixed to the wedge at an inclination relative to the direction in which the fluid travels through the tubular body such that the ultrasound transducer receives only the ultrasound echo from the reflection of a shear wave component off the reflectors in the fluid.

The inclination is such that when the velocities of a longitudinal wave and a shear wave of the ultrasound propagating through the tubular body are equal to or higher than the velocity of the longitudinal wave propagating through the wedge, the incidence angle of the ultrasound transmitted from the wedge into the tubular body is equal to or higher than the critical angle of the longitudinal wave that is determined by the velocity of the longitudinal wave propagating through the wedge and the velocity of the longitudinal wave propagating through the tubular body, and is equal to or lower than the critical angle of the shear wave that is determined by the velocity of the longitudinal wave propagating through the wedge and the velocity of the shear wave propagating through the tubular body.

The inclination also can be such that when the velocities of a longitudinal wave and a shear wave of the ultrasound propagating through the tubular body are equal to or higher than the acoustic velocity in the fluid, the incidence angle of the ultrasound transmitted from the tubular body into the fluid is equal to or higher than the critical angle of the longitudinal wave that is determined by the acoustic velocity propagating through the fluid and the velocity of the longitudinal wave propagating through the tubular body, and is equal to or lower than the critical angle of the shear wave that is determined by the acoustic velocity propagating through the fluid and the velocity of the shear wave propagating through the tubular body.

The wedge can be made of a resin or metal. The tubular body also can be made of a metal or resin. The resin for the wedge can be composed of any of acrylic, epoxy resin, polyvinyl chloride, and polyphenylene sulfide. The resin for the tubular body can be composed of any of polyvinyl chloride, acrylic, FRP, polyethylene, polytetrafluoroethylene, tar epoxy, and mortar. The metal for the wedge can be any of iron, steel, cast iron, stainless, copper, lead, aluminum, and brass. The metal for the tubular body can be any of iron, steel, ductile cast iron, cast iron, stainless, copper, lead, aluminum, and brass.

The inclination is such that an incidence angle of ultrasound pulse at the interface between the wedge and the tubular body is 45 degrees.

Another aspect of the present invention is the method of measuring a flow rate profile of fluid, traveling in the tubular body, based on the frequency of ultrasound changed by the Doppler effect when ultrasound is reflected by the reflectors existing in the fluid. The method can comprise the steps of mounting externally on the tubular body, the wedge previously mentioned, mounting the ultrasound transducer previously mentioned on the wedge at the inclination mentioned previously relative to the direction in which the fluid travels through the tubular body such that the ultrasound transducer only receives the ultrasound echo from the reflection of a shear wave component off the reflectors in the fluid.

### BRIEF DESCRIPTION OF THE DRAWINGS

FIG. 1 shows the structure of the main part illustrating the first embodiment of the present invention.

FIG. 2 shows the relation shown in Publication 5 between the incidence angle of the ultrasound from water into the piping and the energy transmission coefficients of the longitudinal wave and the shear wave in the piping.

FIG. 3 shows an example in which the flow rate to the position along the diameter direction of the piping is measured when the oblique angle of the ultrasound transducer is 15 degrees.

FIG. 4 shows an example in which the flow rate to the position along the diameter direction of the piping is measured when the oblique angle of the ultrasound transducer is 45 degrees.

FIG. 5 shows the comparison, with regards to the measurement errors of the flow rate output, between the first embodiment of the present invention and a conventional technique.

FIG. 6 shows a conventional clamp-on type ultrasound Doppler current flow meter.

FIG. 7 shows sections (A), (B), and (C) that illustrate the mechanism through which a Doppler ultrasound flow meter operates.

FIG. 8 shows the propagation status of an acoustic wave when the acoustic wave propagates in different mediums.

FIG. 9 shows the measurement conditions shown in Publications 2 and 3.

FIG. 10 shows the relationship, shown in Publication 4, between the incidence angle and the transmission rate and the reflectivity.

FIG. 11 shows the relationship, shown in Publication 4, between the incidence angle and the transmission rate and the reflectivity.

FIG. 12 shows the behavior of the ultrasound echo in FIG. 9.

FIG. 13 shows a further enlarged view of FIG. 9.

FIG. 14 shows the flow rate profile for explaining the objective of the present invention.

### DETAILED DESCRIPTION

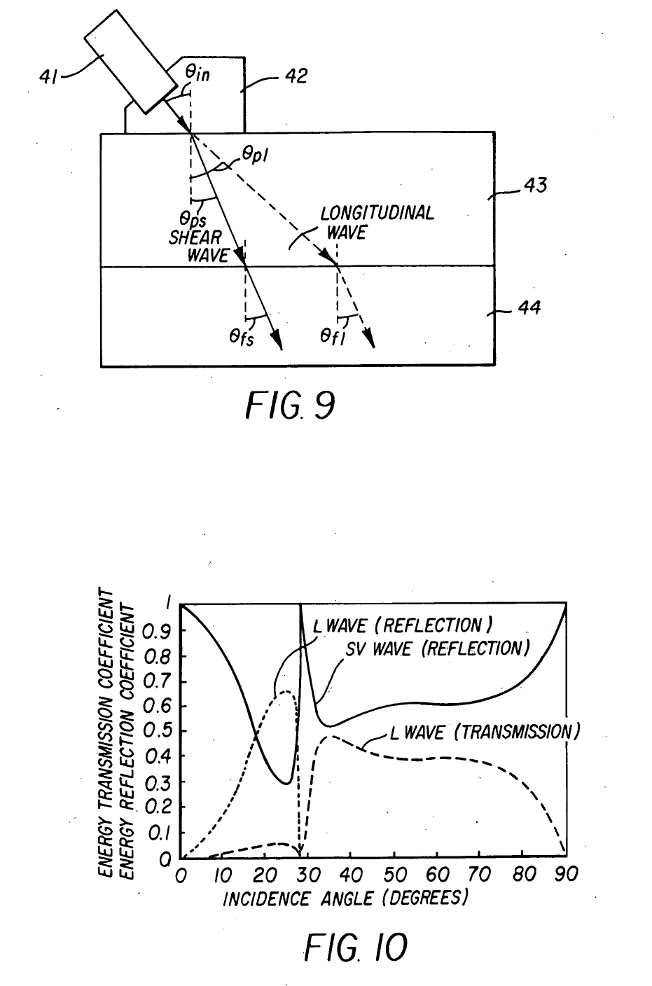

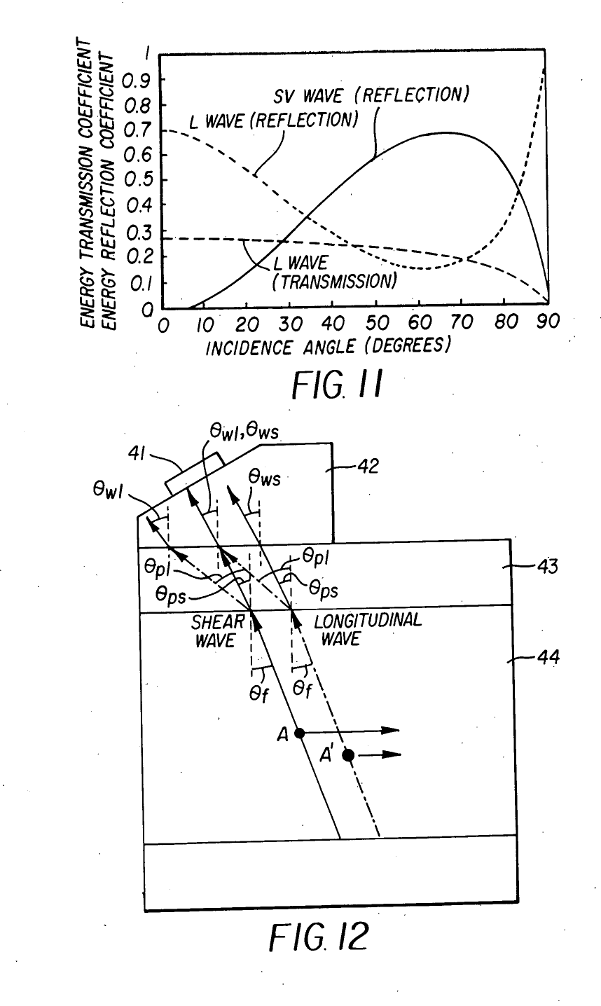

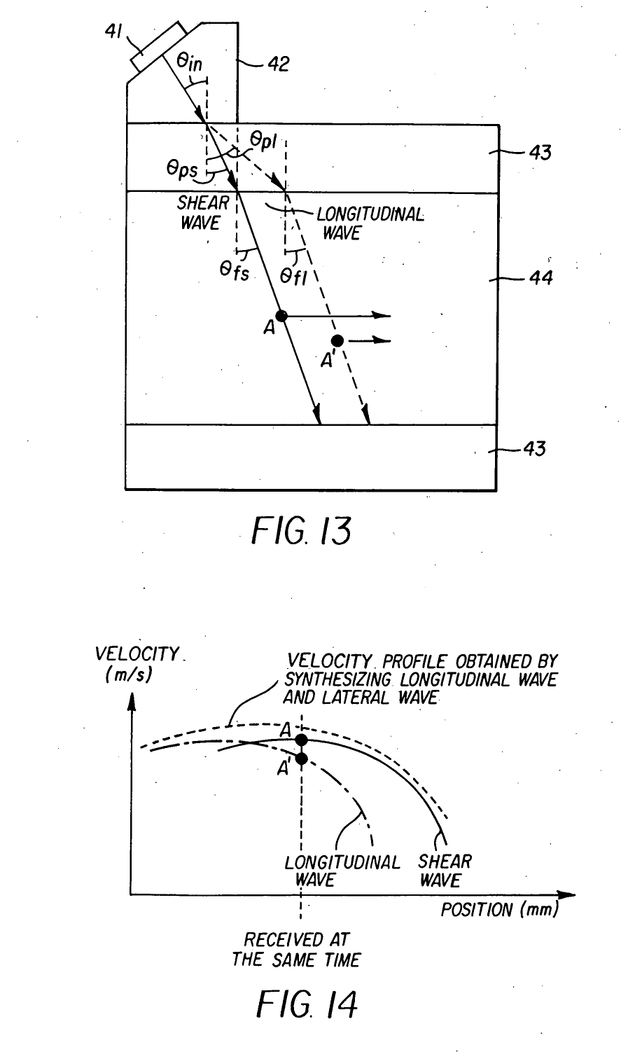

FIG. 1 shows the main part of the first embodiment. Although the structure of FIG. 1 include substantially the same components as included in FIG. 9, FIG. 12, FIG. 13, etc., different reference numerals are used, for clarity. Reference numeral 51 denotes an ultrasound transducer, which generates an acoustic wave. This ultrasound oscillator or transducer 51 can be made of a piezoelectric material, such as PZT (e.g., zircon, lead titanate) and operates both as an ultrasound transmitter/receiver. Reference numeral 52 denotes a wedge made of a resin material in which an acoustic wave can propagate (e.g., acrylic, epoxy resin, polyvinyl chloride, polyphenylene sulfide). The wedge 52 has an inclined plane 52a at the upper end thereof, and the ultrasound transducer 51 can be fixed to the inclined plane 52a with an epoxy adhesive agent or the like. The inclined plane 52a is inclined such that the oblique angle of the ultrasound transducer 51 to the direction perpendicular to the longitudinal direction of the piping 53 (incidence angle of the ultrasound pulse at the interface between the wedge 52 and the piping 53) is equal to θᵢₙ. Reference numeral 54 denotes fluid.

Still referring to FIG. 1, the velocity of sound in the piping 53 is higher than that in the wedge 52, when the wedge 52 is made of acrylic while the piping 53 is made of aluminum, for example, and the fluid 54 is water. The speed of sound in acrylic is about 2,730 m/s, the velocity of the longitudinal wave in aluminum is about 6,420 m/s while the velocity of the shear wave therein is about 3,040 n/s, and the speed of sound in water is about 1,500 m/s. The piping 53 may be made of aluminum or other metal in which an acoustic wave can propagate (e.g., iron, steel, ductile cast iron, stainless steel, copper, lead, brass). There is a critical angle when an ultrasound pulse is transmitted from the wedge 52 into the piping 53 and when an ultrasound pulse is transmitted from the fluid 54 into the piping 53. As is clear from Snell's law, the critical angle of any material has the relation as shown in the following mathematical expression (5):
sin θᵢₙ/c_{w}=sin θₚₗ/cₚₗ=sin θₚₛ/cₚₛ=sin θ_{f}/c_{f} (5),
where c_{w} represents the acoustic velocity in the wedge 52, cₚₗ represents the acoustic velocity of the longitudinal wave in the piping 53, cₚₛ represents the acoustic velocity of the shear wave in the piping 53, c_{f} represents the acoustic velocity in the fluid 54, θᵢₙ represents the oblique angle of the acoustic wave in the wedge 52 (incidence angle to piping 53), θₚₗ represents the angle of the longitudinal wave in the piping 53 (refraction angle), θₚₛ represents the angle of the shear wave in the piping 53 (refraction angle), and θ_{f} represents the incidence angle θ in the fluid 54.

When the wedge 52 is made of acrylic, the piping 53 is made of aluminum, and the fluid 54 is water, then the critical angle of the longitudinal wave is 25.2 degrees, while the critical angle of the shear wave is 63.9 degrees when ultrasound is transmitted from the wedge 52 into the piping 53. Thus, when the oblique angle θᵢₙ of the ultrasound transducer 51 (incidence angle at the interface between the wedge 52 and the piping 53) is within the above critical angle range (i.e., 25.2 degrees≦θin≦63.9 degrees), only the shear wave propagates in the piping 53 because the longitudinal wave is totally reflected at the interface between the wedge 52 and the piping 53. As a result, only the ultrasound along one measurement line caused by the shear wave in the piping 53 is transmitted into water. Subsequently, only the ultrasound echo from the reflectors in water reflected by the shear wave component is received. Specifically, the ultrasound transducer 51 does not receive the ultrasound echo caused by the longitudinal wave, thus reducing the acoustic noise included in the measured flow rate. This improves the measurement accuracy of the flow rate profile and enables the flow rate to be calculated with a higher accuracy.

Next, an example will be specifically described in which the wedge 52 shown in FIG. 1 produces an acoustic wave of an incidence angle θᵢₙ of 45 degrees. When the acoustic wave propagates from the wedge 52 into the aluminum piping 53, the above incidence angle θᵢₙ exceeds 25.2 degrees (which is the critical angle of the longitudinal wave). Thus, the longitudinal wave is totally reflected at the interface between the wedge 52 and the piping 53, and does not propagate in the piping 53. On the other hand, the shear wave propagates in the piping 53 with the refraction angle of 51.9 degrees.

Next, when the acoustic wave is transmitted from the piping 53 into the fluid 54 (which is water), then only the longitudinal wave exits into the water. As a result, the longitudinal wave propagates in water at a refraction angle (θ_{fs} in FIG. 1) of 22.8 degrees along one measurement line. The longitudinal wave reflected off the reflector, i.e., the ultrasound echo, is also transmitted into the piping 53 at an incidence angle of 22.8 degrees.

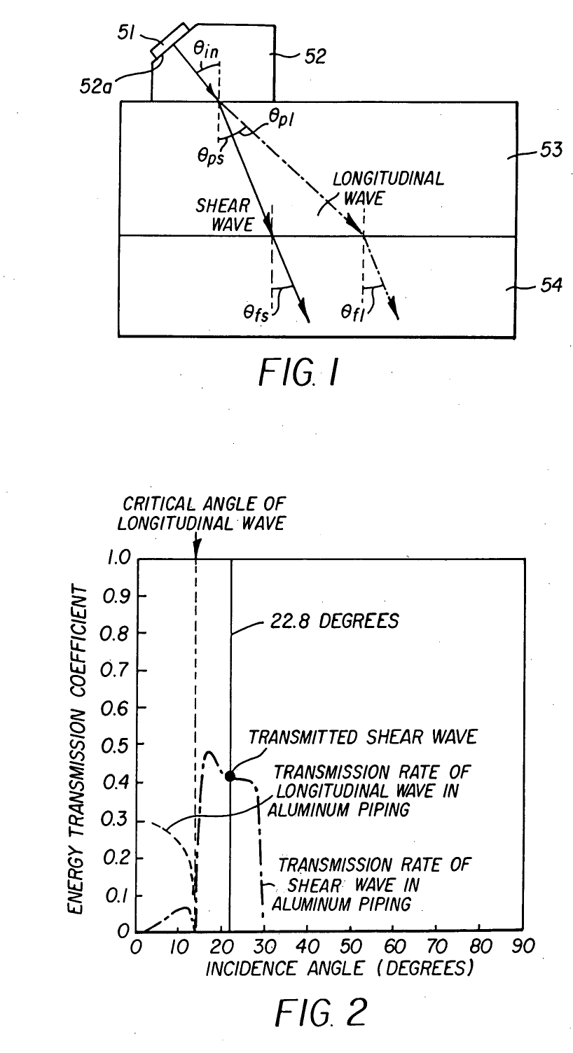

With regards to the transmission of the acoustic wave from water into the aluminum piping, data is shown in FIG. 2, as provided in a publication (hereafter Publication 5) entitled ACOUSTIC WAVE” by Cordon S. Kino. FIG. 2 shows the relation between the incidence angle of an ultrasound wave from water into the piping and the energy transmission coefficient (transmission rate) of the longitudinal wave and the shear wave in the piping.

According to FIG. 2, the incidence angle to the piping 53 of 22.8 degrees is equal to or higher than the critical angle of the longitudinal wave, and thus, the longitudinal wave is totally reflected at the interface between water and the piping 53. Specifically, the longitudinal wave does not propagate in the piping 53. Thus, the piping 53 has therein only one measurement line of the ultrasound echo produced by the shear wave and the ultrasound transducer 51 receives the ultrasound echo of this shear wave, thus reducing the conventional acoustic noise caused by the longitudinal wave.

As described above, the measurement accuracy of the flow rate profile can be improved over conventional cases by improving the oblique angle of the ultrasound transducer 51 (incidence angle to the piping 53) to eliminate the longitudinal wave in the piping 53.

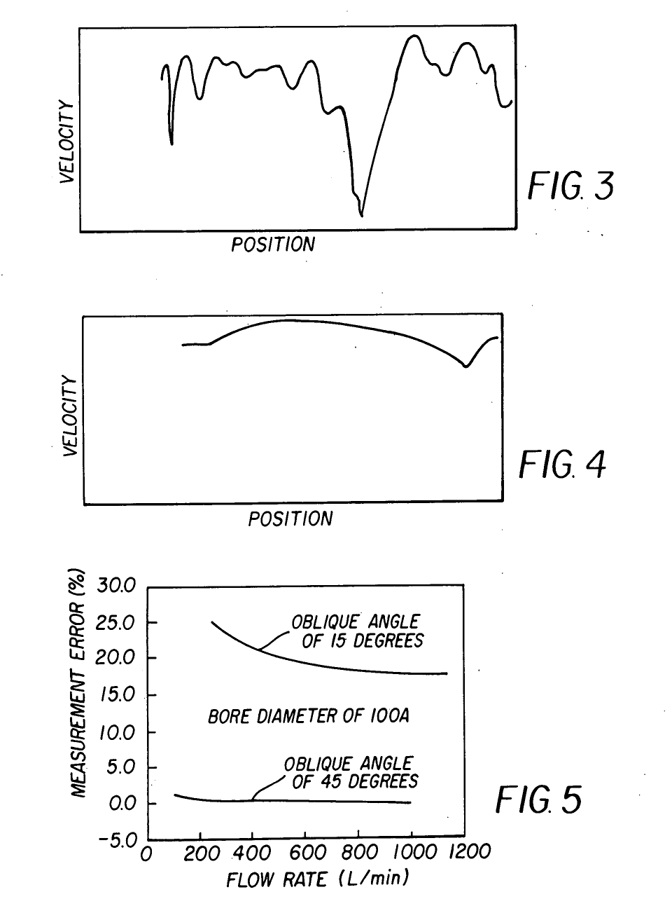

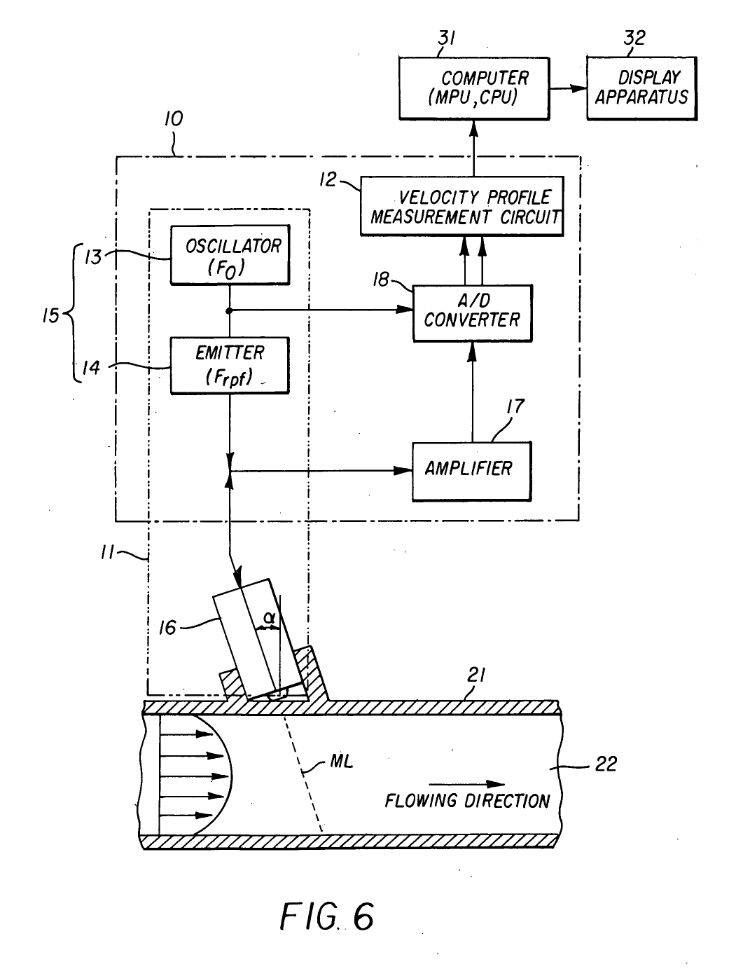

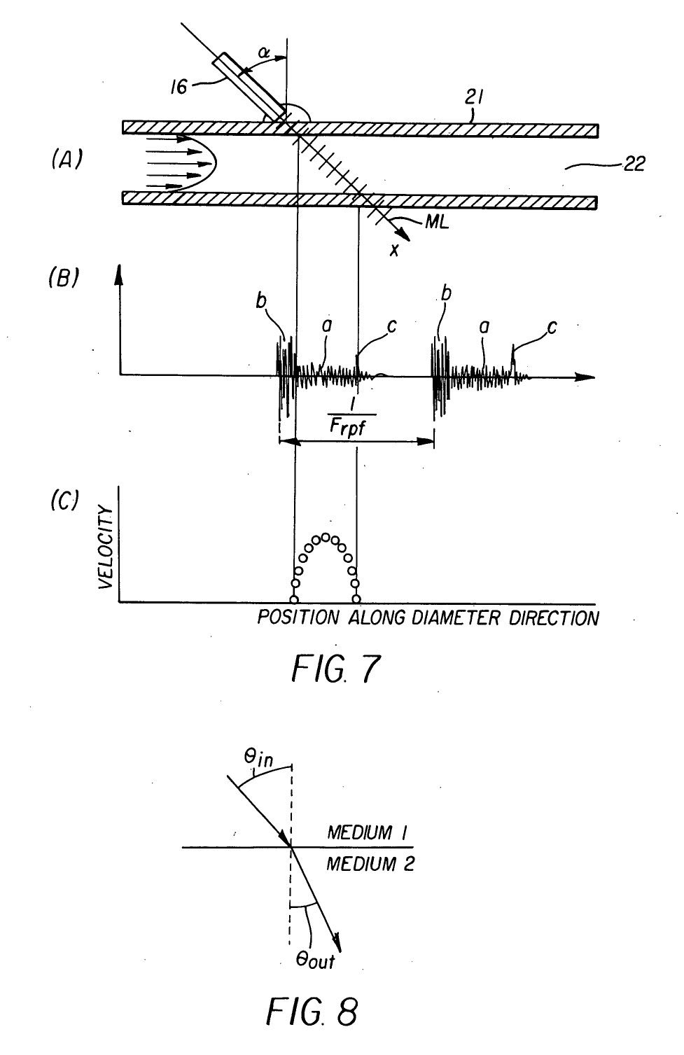

FIGS. 3 and 4 show examples in which the flow rate to the position along the diameter direction of the piping 53 is measured when the oblique angle of the ultrasound transducer 51 is 15 degrees (FIG. 3) and when the oblique angle of the ultrasound transducer 51 is 45 degrees (FIG. 4). When the oblique angle of the ultrasound transducer 51 is set at 45 degrees, which is equal to or higher than the critical angle of the longitudinal wave, when the ultrasound is transmitted from the wedge 52 into the piping 53 (25.2 degrees) at an angle that is equal to or lower than the critical angle of the shear wave (63.9 degrees), then appropriate measurement values as shown in FIG. 4 are obtained according to which the flow rate is continuously changed depending on the position along the diameter direction. When the oblique angle is set at 15 degrees, the piping 53 has therein both the longitudinal wave and the shear wave, and thus the ultrasound echo is received by the ultrasound transducer 51. Because the ultrasound echo includes a large amount of acoustic noise, the measurement values of flow rate profile becomes unstable, which deteriorates the measurement accuracy.

FIG. 5 shows the comparison, with regards to the measurement errors that occurred when the flow rate output of an electromagnetic flow meter was measured based on the flow rate profile, between a case in which the oblique angle of the ultrasound transducer 51 is similarly provided to be 45 degrees according to this embodiment, and a case in which the oblique angle of the ultrasound transducer 51 is provided to be 15 degrees, as in conventional cases. As can be seen from FIG. 5, this embodiment also significantly improves the measurement errors when compared to conventional cases.

In the second embodiment of the present invention, only the longitudinal wave element of the ultrasound echo propagating in the piping 53 after being reflected by the reflector in the fluid 54 is eliminated. It is assumed that, when the fluid 54 is water, for example, the critical angle of the longitudinal wave in the ultrasound echo transmitted into the aluminum piping 53 after being reflected by the reflector in water is 13.5 degrees while the critical angle of the shear wave is 29.6 degrees when the acoustic wave in water is 1500 m/s.

Thus, when the acoustic wave from the piping 53 into water has an incidence angle that is equal to or higher than 13.5 degrees and that is equal to or lower than 29.6 degrees, then only the shear wave element is transmitted into the piping 53 and the longitudinal wave element is eliminated when the ultrasound echo is transmitted from water into the piping 53, thus reducing the acoustic noise caused by the longitudinal wave. As a result, the ultrasound transducer 51 receives only the ultrasound echo of the shear wave in the piping 53, and this allows the piping 53 to have reduced acoustic noise caused by the longitudinal wave, provides the measurement of a flow rate profile with a higher accuracy, and improves the accuracy of the measurement of a flow meter.

The wedge also can be made of a metal in which an acoustic wave can propagate (e.g., iron, steel, cast iron, stainless steel, copper, lead, aluminum, brass), and the piping may be made of a resin in which an acoustic wave can propagate (e.g., polyvinyl chloride, acrylic, FRP, polyethylene, polytetrafluoroethylene (also known as Teflon®), tar epoxy, mortar).

According to the present invention, The longitudinal wave element of the ultrasound that is transmitted from an ultrasound transducer and that propagates in the tubular body or from the wedge to the tubular body can be eliminated. Thus, the fluid has therein only ultrasound along one measurement line, caused by the shear wave in the tubular body. As a result, only the ultrasound echo caused by reflection of the shear wave off the reflector in the fluid appears. Thus, the ultrasound echo caused by the longitudinal wave is not received by the ultrasound transducer, thus reducing the acoustic noise.

Given the disclosure of the present invention, one versed in the art would appreciate that there may be other embodiments and modifications within the scope and spirit of the present invention. Accordingly, all modifications and equivalents attainable by one versed in the art from the present disclosure within the scope and spirit of the present invention are to be included as further embodiments of the present invention. The scope of the present invention accordingly is to be defined as set forth in the appended claims.

The disclosure of the priority applications, JP 2003-396755, in its entirety, including the drawings, claims, and the specifications thereof, is incorporated herein by reference.

1. An apparatus for measuring a velocity profile of fluid traveling through a tubular body made of material that allows an acoustic wave to propagate therethrough, based on the frequency of ultrasound changed by the Doppler effect when ultrasound is reflected off reflectors existing in the fluid, comprising:
a wedge for externally mounting to the tubular body;
an ultrasound oscillator mounted to the wedge,
wherein the wedge is made of material that allows an acoustic wave to propagate therethrough,
wherein the ultrasonic oscillator is fixed to the wedge at an inclination relative to the direction in which the fluid travels through the tubular body such that the ultrasound oscillator receives only the ultrasound echo from the reflection of a shear wave component off the reflectors in the fluid.

2. An apparatus according to claim 1, wherein when the velocities of a longitudinal wave and a shear wave of the ultrasound propagating through the tubular body are equal to or higher than the velocity of the longitudinal wave propagating through the wedge, the incidence angle of the ultrasound transmitted from the wedge into the tubular body is equal to or higher than the critical angle of the longitudinal wave that is determined by the velocity of the longitudinal wave propagating through the wedge and the velocity of the longitudinal wave propagating through the tubular body, and is equal to or lower than the critical angle of the shear wave that is determined by the velocity of the longitudinal wave propagating through the wedge and the velocity of the shear wave propagating through the tubular body.

3. An apparatus according to claim 1, when the velocities of a longitudinal wave and a shear wave of the ultrasound propagating through the tubular body are equal to or higher than the acoustic velocity in the fluid, the incidence angle of the ultrasound transmitted from the tubular body into the fluid is equal to or higher than the critical angle of the longitudinal wave that is determined by the acoustic velocity propagating through the fluid and the velocity of the longitudinal wave propagating through the tubular body, and is equal to or lower than the critical angle of the shear wave that is determined by the acoustic velocity propagating through the fluid and the velocity of the shear wave propagating through the tubular body.

4. An apparatus according to claim 2, wherein in that the wedge is made of a resin or metal.

5. An apparatus according to claim 3, characterized in that the wedge is made of a resin or metal.

6. An apparatus according to claim 2, wherein the tubular body is made of a metal or resin.

7. An apparatus according to claim 3, wherein the tubular body is made of a metal or resin.

8. An apparatus according to claim 4, wherein the tubular body is made of a metal or resin.

9. An apparatus according to claim 5, wherein the tubular body is made of metal or resin.

10. An apparatus according to claim 4, wherein the resin is composed of one or more material selected from acrylic, epoxy resin, polyvinyl chloride, and polyphenylene sulfide.

11. An apparatus according to claim 5, wherein the resin is composed of one or more material selected from acrylic, epoxy resin, polyvinyl chloride, and polyphenylene sulfide.

12. An apparatus according to claim 6, wherein the resin is composed of one or more material selected from polyvinyl chloride, acrylic, FRP, polyethylene, polytetrafluoroethylene, tar epoxy, and mortar.

13. An apparatus according to claim 7, wherein the resin is composed of one or more material selected from polyvinyl chloride, acrylic, FRP, polyethylene, polytetrafluoroethylene, tar epoxy, and mortar.

14. An apparatus according to claim 8, wherein the resin is composed of one or more material selected from polyvinyl chloride, acrylic, FRP, polyethylene, polytetrafluoroethylene, tar epoxy, and mortar.

15. An apparatus according to claim 9, wherein the resin is composed of one or more material selected from polyvinyl chloride, acrylic, FRP, polyethylene, polytetrafluoroethylene, tar epoxy, and mortar.

16. An apparatus according to claim 10, wherein the metal is composed of one or more material selected from iron, steel, cast iron, stainless, copper, lead, aluminum, and brass.

17. An apparatus according to claim 11, wherein the metal is composed of one or more material selected from iron, steel, cast iron, stainless, copper, lead, aluminum, and brass.

18. An apparatus according to claim 12, the metal is composed of one or more material selected from iron, steel, ductile cast iron, cast iron, stainless, copper, lead, aluminum, and brass.

19. An apparatus according to claim 13, the metal is composed of one or more material selected from iron, steel, ductile cast iron, cast iron, stainless, copper, lead, aluminum, and brass.

20. An apparatus according to claim 14, the metal is composed of one or more material selected from iron, steel, ductile cast iron, cast iron, stainless, copper, lead, aluminum, and brass.

21. An apparatus according to claim 15, the metal is composed of one or more material selected from iron, steel, ductile cast iron, cast iron, stainless, copper, lead, aluminum, and brass.

22. (canceled)

23. A method of measuring a velocity profile of fluid, traveling in a tubular body made of a material that allows an acoustic wave to propagate therethrough, based on the frequency of ultrasound changed by the Doppler effect when ultrasound is reflected off reflectors existing in the fluid, comprising the steps of:
mounting externally on the tubular body, a wedge made of a material that allows an acoustic wave to propagate through;
mounting an ultrasound oscillator on the wedge at an inclination relative to the direction in which the fluid travels through the tubular body such that the ultrasound oscillator receives only the ultrasound echo from the reflection of a shear wave component off the reflectors in the fluid.
